(19) United States
(12) Patent Application Publication
(10) Pub. No.: US 2003/0028045 A1
(43) Pub. Date: Feb. 6, 2003

(54) Method for producing chlorocarboxylic acid chlorides

(76) Inventors: Armin Stamm, Nieder-Olm (DE); Heinz-Josef Kneuper, Niederkirchen (DE); Jochem Henkelmann, Mannheim (DE); Theodor Weber, Ludwigshafen (DE); Ralph Busch, Worms (DE)

Correspondence Address: KEIL & WEINKAUF, 1350 CONNECTICUT AVENUE, N.W., WASHINGTON, DC, 20036, US

(21) Appl. No.: 10/204,965

(22) PCT Filed: Feb. 28, 2001
(86) PCT No.: PCT/EP01/02239

(51) Int. Cl. C07C 253/30; C07C 51/58

(52) U.S. Cl. 558/440; 562/857

(57) ABSTRACT

A process for preparing chlorocarbonyl chlorides of the formula (I)

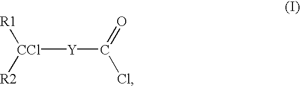

in which

R¹ and R² independently of one another

are a hydrogen atom, a carbon-containing organic radical, a halogen, a nitro or a cyano group,

and Y

is an alkylene chain having 1 to 10 carbon atoms in the chain, which is unsubstituted or substituted by carbon-containing organic radicals, halogen, nitro and/or cyano groups, where the alkylene chain may be interrupted by an ether, a thioether, a tertiary amino or a keto group,

where the carbon-containing organic radicals of Y and/or R¹ and/or R² may be attached to one another forming a nonaromatic system, by reacting a lactone of the formula (II)

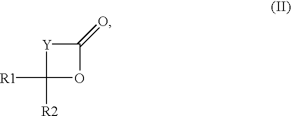

in which R¹, R² and Y are as defined above, with hydrogen chloride and phosgene in the presence of a catalyst, which comprises introducing hydrogen chloride before and/or during the addition of phosgene, where the introduction of hydrogen chloride is started only when a temperature of at least 60° C. has been reached and a pyridine compound is used as catalyst is described.

The invention relates to a process for preparing chlorocarbonyl chlorides of the formula (I)

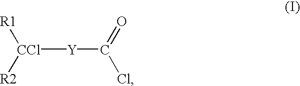

in which

R¹ and R² independently of one another

are a hydrogen atom, a carbon-containing organic radical, a halogen, a nitro or a cyano group,

and Y

is an alkylene chain having 1 to 10 carbon atoms in the chain, which is unsubstituted or substituted by carbon-containing organic radicals, halogen, nitro and/or cyano groups, where the alkylene chain may be interrupted by an ether, a thioether, a tertiary amino or a keto group,

where the carbon-containing organic radicals of Y and/or R¹ and/or R² may be attached to one another forming a nonaromatic system,

by reacting a lactone of the formula (II)

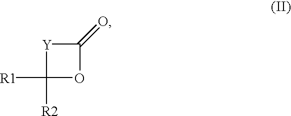

in which R¹, R² and Y are as defined above, with hydrogen chloride and phosgene in the presence of a catalyst.

Chlorocarbonyl chlorides are important reactive intermediates for preparing pharmaceutically and agrochemically active compounds.

Chlorocarbonyl chlorides can be prepared, for example, by reacting the corresponding lactones with chlorinating agents in the presence of a catalyst. Typical chlorinating agents are phosgene and thionyl chloride, since the coproducts formed with them are exclusively gaseous substances (CO₂ or SO₂ and HCl)

If the chlorinating agent used is phosgene, various catalyst systems are generally employed. U.S. Pat. No. 2,778,852 mentions, as suitable catalysts for the phosgenation of γ-butyrolactone, γ- and δ-valerolactone, pyridines, tertiary amines, heavy metals and acids, such as sulfuric acid, phosphoric acid, phosphorus chloride, phosphorus oxychloride, aluminum chloride, sulfuryl chloride and chlorosulfonic acid.

DE-A 197 53 773 describes the phosgenation of aliphatic lactones in the presence of a urea compound as catalyst, with simultaneous introduction of hydrogen chloride.

EP-A 0 413 264 and EP-A 0 435 714 disclose the phosgenation of lactones in the presence of a phosphine oxide as catalyst, where, according to the teaching of EP-A 0 413 264, the simultaneous introduction of hydrogen chloride is described as being advantageous.

EP-A 0 583 589 describes the phosgenation of benzo-fused lactones in the presence of an organic nitrogen compound, such as, for example, a quaternary ammonium salt, an amine, a nitrogen heterocycle, a urea compound, a guanidine compound or a formamide as catalyst, with simultaneous introduction of hydrogen chloride.

EP-A 0 253 214 mentions the phosgenation of aliphatic lactones in the presence of a quaternary ammonium salt as catalyst, specifically trimethylbenzylammonium chloride, N,N-dimethylpiperidinium chloride and dimethylmorpholinium chloride, and the simultaneous introduction of hydrogen chloride is described as being particularly advantageous. In the reaction of δ-valerolactone with phosgene and hydrogen chloride at 175-180° C. in the presence of N,N-dimethylpiperidiniumchloride, 5-chlorovaleryl chloride was obtained in a purity of 98.1% and a yield of 76.2%.

According to the invention, it was realized that in the preparation of chlorocarbonyl chlorides, in particular 5-chlorovaleryl chlorides, in some cases considerable problems are encountered when the corresponding lactones are phosgenated in the presence of the abovementioned catalysts. Since some of the lactones to be used as starting materials, in particular the 6-membered systems, are highly labile under the customary phosgenation conditions, under the conditions described in the prior art, frequently a high proportion of oligomers is formed, resulting in a considerable increase in viscosity, and the reaction mixture may even solidify. Because of this, there is a considerable safety risk owing to apparatus and pipelines being blocked during the synthesis and the usually distillative work-up. Owing to the increased formation of oligomers, the yield of the product of value is reduced significantly and the proportion of residue requiring disposal is increased considerably.

A further disadvantage of using customary phosgenation conditions was found to be that in many cases the reaction mixtures foam very strongly, owing to which the reaction has to be throttled back considerably or even terminated. The increased formation of foam, too, is a considerable safety risk.

It is an object of the present invention to develop a process for preparing chlorocarbonyl chlorides by reacting the corresponding lactones with chlorinating agents, which process no longer has the known disadvantages, permits the reaction to be carried out safely and makes available the chlorocarbonyl chlorides in high yield and high purity.

We have found that this object is achieved by a process for preparing chlorocarbonyl chlorides of the formula (I)

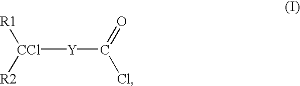

in which

R¹ and R² independently of one another

are a hydrogen atom, a carbon-containing organic radical, a halogen, a nitro or a cyano group,

and Y

is an alkylene chain having 1 to 10 carbon atoms in the chain, which is unsubstituted or substituted by carbon-containing organic radicals, halogen, nitro and/or cyano groups, where the alkylene chain may be interrupted by an ether, a thioether, a tertiary amino or a keto group,

where the carbon-containing organic radicals of Y and/or R¹ and/or R² may be attached to one another forming a nonaromatic system, by reacting a lactone of the formula (II)

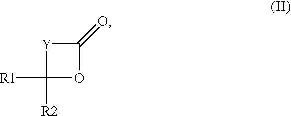

in which R¹, R² and Y are as defined above, with hydrogen chloride and phosgene in the presence of a catalyst, which comprises introducing hydrogen chloride before and/or during the addition of phosgene, where the introduction of hydrogen chloride is started only when a temperature of at least 60° C. has been reached and a pyridine compound is used as catalyst.

The catalyst used in the process according to the invention is a pyridine compound of the formula (III)

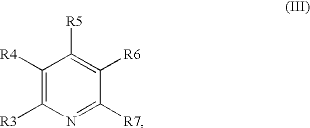

in which the radicals R³ to R⁷ independently of one another are hydrogen, a carbon-containing organic radical, halogen, a nitro or a cyano group.

A carbon-containing organic radical is to be understood as meaning an unsubstituted or substituted, aliphatic, aromatic or araliphatic radical having 1 to 20 carbon atoms. This radical may contain one or more heteroatoms, such as oxygen, nitrogen or sulfur, for example —O—, —S—, —NR—, —CO— and/or —N═ in aliphatic or aromatic systems, and/or be substituted by one or more functional groups containing, for example, oxygen, nitrogen, sulfur and/or halogen, such as, for example, by fluorine, chlorine, bromine, iodine and/or a cyano group. If the carbon-containing organic radical contains one or more heteroatoms, it can also be attached via a heteroatom. Thus, for example, ether, thioether and tertiary amino groups are also included. Preferred examples of the carbon-containing organic radical which may be mentioned are C₁- to C₂₀-alkyl, in particular C₁- to C₆-alkyl, C₆- to C₁₀-aryl, C₇- to C₂₀-aralkyl, in particular C7- to C₁₀-aralkyl, and C7- to C₂₀-alkaryl, in particular C7- to C₁₀-alkaryl.

It is furthermore possible that two adjacent radicals are attached to one another forming a nonaromatic or aromatic system.

Halogens which may be mentioned are fluorine, chlorine, bromine and iodine.

Preference is given to pyridine compounds (III) in which R³ to R⁷ independently of one another are hydrogen, C₁- to C₆-alkyl, C6- to C₁₀-aryl, C7- to C₁₀-aralkyl or C7- to C₁₀-alkaryl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, l-ethyl-2-methylpropyl, phenyl, 2-methylphenyl (o-tolyl), 3-methylphenyl (m-tolyl), 4-methylphenyl (p-tolyl), naphthyl or benzyl.

Particular preference is given to hydrogen and C₁- to C4-alkyl, such as, for example, methyl, ethyl, propyl, 1-methylethyl, butyl, 1-methylpropyl, 2-methylpropyl and 1,1-dimethylethyl, in particular to hydrogen and methyl.

Pyridine compounds suitable for use in the process according to the invention are, for example, pyridine, 2-methylpyridine (α-picoline), 3-methylpyridine (β-picoline), 4-methylpyridine (γ-picoline), 2,3-dimethylpyridine, 2,4-dimethylpyridine, 2,5-dimethylpyridine, 2,6-dimethylpyridine, 3,4-dimethylpyridine, 3,5-dimethylpyridine, 2,3,4-trimethylpyridine, 2,3,5-trimethylpyridine, 2,3,6-trimethylpyridine, 2,4,6-trimethylpyridine, 3,4,5-trimethylpyridine, 2,3,4,5-tetramethylpyridine, 2,3,4,6-tetramethylpyridine, 2,3,5,6-tetramethylpyridine, 2,3,4,5,6-pentamethylpyridine, 2-ethylpyridine, 3-ethylpyridine, 4-ethylpyridine, 2-propylpyridine, 3-propylpyridine, 4-propylpyridine, 2-butylpyridine, 3-butylpyridine, 4-butylpyridine, 2-butylpyridine, 3-butylpyridine, 4-butylpyridine, 2-phenylpyridine, 3-phenylpyridine, 4-phenylpyridine, quinoline and isoquinoline.

Preference is given to pyridine and the mono-C₁-C₄-alkylpyridines, and very particular preference is given to the monomethylpyridines 2-methylpyridine (α-picoline), 3-methylpyridine (β-picoline) and 4-methylpyridine (γ-picoline), in particular to 3-methylpyridine (β-picoline).

In the process according to the invention, the pyridine compound (III) is generally employed in a concentration of from 0.1 to 20 mol %, preferably from 0.1 to 10 mol %, particularly preferably from 0.5 to 10 mol %, in particular from 1 to 6 mol %, based on the lactone (II).

Furthermore, the introduction of hydrogen chloride before and/or during the addition of phosgene is essential in the process according to the invention. Decisive for the successful addition of hydrogen chloride is the temperature at which the introduction is started. In the process according to the invention, hydrogen chloride is introduced only when the temperature of the reaction mixture has reached at least 60° C. Preferably, the hydrogen chloride is introduced only when a temperature of at least 80° C., particularly preferably 100° C. and very particularly preferably at least 110° C., in particular at least 120° C., has been reached.

As the temperature at which the introduction of hydrogen chloride is started increases, the proportion of oligomeric and polymeric byproducts generally decreases. Thus, the product yield which can be obtained is increased and the amount of residue which has to be disposed of is reduced.

At a temperature below 60° C., i.e. not within the range according to the invention, the reaction mixture generally becomes viscous or even solid, and the desired chlorination reaction stops.

The hydrogen chloride is generally added in gaseous form and can take place (i) before the addition of phosgene, (ii) before and during the addition of phosgene or (iii) during the addition of phosgene. Preference is given to the variants (ii) “before and during” and (iii) “during” the addition of phosgene.

If no hydrogen chloride is added before or during the reaction with phosgene, or if the hydrogen chloride is introduced only after the addition of phosgene has started, the yield that can be obtained decreases considerably, and the risk of the reactor content becoming viscous or even solidifying increases significantly. The negative effect is generally greater the more phosgene has already been added.

Of course, it is also possible to continue with the introduction of hydrogen chloride after the addition of phosgene has ended. In certain cases, this so-called after-reaction is even advantageous in order to bring the reaction to completion. Moreover, unreacted excess phosgene is eliminated from the reaction solution.

The total amount of hydrogen chloride added is generally of minor importance. Typically, a total of from 0.5 to 2 mol, preferably from 0.5 to 1.5 mol, of hydrogen chloride per mole of lactone 10 (II) are added.

In the process according to the invention, the reaction with phosgene can be carried out at a temperature of 60-200° C., preferably 100-200° C., particularly preferably 110-150° C. It is 15 generally carried out at an absolute pressure of 0.01-5 Mpa, preferably 0.05 to 0.2 MPa and particularly preferably at atmospheric pressure. The phosgene can be metered in in gaseous or liquid form. The addition of gaseous phosgene is preferred.

In the process according to the invention, the total amount of phosgene introduced is generally from 0.8 to 1.5 mol, preferably from 0.9 to 1.2 mol, per mole of lactone (II).

The chlorocarbonyl chlorides which can be prepared by the process according to the invention have the formula (I)

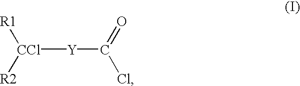

in which R¹ and R² independently of one another are a hydrogen atom, a carbon-containing organic radical, a halogen, a nitro or a cyano group.

The carbon-containing organic radical is defined as in the description of the pyridine compound (III). Preferred examples of the carbon-containing organic radical which may be mentioned are C₁- to C₂₀-alkyl, in particular C₁- to C₆-alkyl, C₆- to C₁₀-aryl, C₇- to C₂₀-aralkyl, in particular C₇- to C₁₀-aralkyl, and C7- to C₂₀-alkaryl, in particular C7- to C₁₀-alkaryl.

Halogens which may be mentioned are fluorine, chlorine, bromine and iodine.

Preference is given to the chlorocarbonyl chlorides (I) in which R¹ and R² independently of one another are hydrogen, C₁- to C₆-alkyl, C₆- to C₁₀-aryl, C₇- to C₁₀-aralkyl or C₇- to C₁₀-alkaryl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, phenyl, 2-methylphenyl (o-tolyl), 3-methylphenyl (m-tolyl), 4-methylphenyl (p-tolyl), naphthyl or benzyl. Particular preference is given to hydrogen and C₁- to C₄-alkyl in particular to hydrogen.

Y is an alkylene chain having 1 to 10 carbon atoms in the chain, which is unsubstituted or substituted by carbon-containing organic radicals, halogen, nitro and/or cyano groups, where the alkylene chain may be interrupted by an ether (—O—), thioether (—S—), tertiary amino (—NR—) or keto (—CO—) group. The carbon-containing organic radicals and halogen are defined as above.

Examples of the radical Y which may be mentioned are the alkylenes (CH₂)ₙ where n is 1 to 10, where one or more, if appropriate all, hydrogen atoms may be replaced by C₁- to C₆-alkyl, C₆- to C₁₀-aryl, C₇- to C₁₀-aralkyl and/or C7- to C₁₀-alkaryl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, phenyl, 2-methylphenyl (o-tolyl), 3-methylphenyl (m-tolyl), 4-methylphenyl (p-tolyl), naphthyl or benzyl.

It is also possible for the organic radicals R¹ and/or R² and/or of Y to be attached to one another, forming a nonaromatic system. An example which may be mentioned is hexahydrophthalide.

Preference is given to the chlorocarbonyl chlorides (I), in which Y is an alkylene chain having 2 to 8 and particularly preferably 2 to 4 carbon atoms in the chain, which is unsubstituted or substituted by carbon-containing organic radicals, halogen, nitro and/or cyano groups.

Very particular preference is given to the chlorocarbonyl chlorides (I) in which the unsubstituted or substituted alkylene chain contains 3 carbon atoms in the chain, since in particular the 6-membered lactones are, compared to their lower and higher homologues, considerably more labile and have an increased tendency to form oligomeric and polymeric byproducts under the phosgenation conditions. The chlorocarbonyl chlorides (I) which are very particularly preferred for the process according to the invention are thus 5-chlorovaleryl chloride (5-chloropentanoyl chloride) and its derivatives, in particular 5-chlorovaleryl chloride.

The lactones to be used as starting materials have the formula (II)

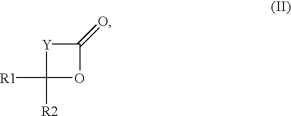

in which R¹, R² and Y are as defined above. It is, of course, also possible to use mixtures of different lactones. Very particular preference is given to using δ-valerolactone and its derivatives, in particular δ-valerolactone.

Suitable reactors for the phosgenation are, in principle, the apparatus which are described in the specialist literature for gas/liquid and liquid/liquid reactions. To achieve a high space/time yield, intensive mixing of the solution which contains the lactone (II) and the pyridine compound (III) and the added phosgene is important. Non-limiting examples which may be mentioned are stirred tanks, stirred-tank batteries, reaction columns operated in countercurrent mode, tubular reactors (preferably with internals), bubble columns and loop reactors.

The process is preferably carried out in the absence of a solvent. However, it is possible to add a solvent which is inert to the phosgene used. Inert solvents are, for example, aromatic hydrocarbons, such as toluene, chlorobenzene, o-, m- or p-dichlorobenzene, o-, m- or p-xylene, cyclic carbonates, such as ethylene carbonate or propylene carbonate, the corresponding chlorocarbonyl chloride target product or mixtures thereof. If solvents are employed, preference is given to using the chlorocarbonyl chloride target product. The addition of a solvent may be advantageous, for example, when lactones (II) are used which have a relatively high molecular weight, are viscous or are solid under the reaction conditions.

The process according to the invention can be carried out batchwise or continuously.

a) Batchwise

In the batchwise preparation, the reaction mixture, comprising the lactone (II), the pyridine compound (III) and, if appropriate, a solvent, is generally initially charged in a reaction apparatus, for example a stirred tank, and mixed intensively. The reaction mixture is then heated, and introduction of hydrogen chloride can be started at a temperature of at least 60° C. If appropriate, the reaction mixture is initially heated without addition of hydrogen chloride gas to a temperature of at least 100° C. or more, and hydrogen chloride and phosgene are then added, for example together. After the addition of phosgene has ended, the reaction solution is allowed to react for a further few minutes to a few hours, during which more hydrogen chloride may be introduced. The after-reaction can be carried out in the reaction apparatus or else in a downstream vessel.

b) Continuously

Reaction apparatus suitable for the continuous process are, for example, stirred tanks, stirred-tank batteries or reaction columns operated in the countercurrent mode. At the beginning of the continuous process, in general a solvent (for example the corresponding chlorocarbonyl chloride target product) and the pyridine compound (III) are initially charged, the system is brought to the desired temperature and gaseous hydrogen chloride is added.

Subsequently, at a temperature of at least 100° C., the continuous introduction of lactone (II), which generally contains further pyridine compound (III) and which may, if appropriate, be dissolved in a solvent, and the continuous addition of liquid or gaseous phosgene is started. In general, more hydrogen chloride is introduced in parallel. After the reactor content has been converted into the chlorocarbonyl chloride, the amounts of lactone (II) and phosgene are adjusted such that both are introduced in essentially equimolar amounts. An amount of the reaction volume which corresponds to the amount that is added is discharged from the reaction apparatus, for example via a means to maintain the level or via an overflow.

Preferably, the reaction solution is introduced into a further vessel for after-reaction.

Alternatively, it is possible to add hydrogen chloride separately to the lactone (II), in a reaction apparatus which is upstream from the phosgenation reactor, if appropriate at a lower temperature, but at at least 60° C.

In general, it is then advantageous to flush out (strip) unreacted phosgene or hydrogen chloride from the reaction solution, for example by passing through a gas which is chemically inert to the reaction solution, such as nitrogen.

Unreacted phosgene which, for example, escapes from the reactor even during the synthesis step and/or which is flushed out by subsequent stripping can advantageously be trapped and reused. Suitable traps are, for example, cold traps in which the phosgene condenses out.

The reaction solution originating from the reaction between the lactone (II) and phosgene can be worked up by customary methods. Preference is given to distillative work-up, and the optional stripping can take place upstream of or in the distillation column.

It is possible and, if appropriate, advantageous to recycle some or all of the fraction, obtained during distillative work-up, which contains the pyridine compound (III). Depending on the boiling points of the chlorocarbonyl chloride (I) and the pyridine compound (III), the pyridine compound (III) can be separated off, for example, before or after the product of value, via the top. If the process is carried out with recycling of the pyridine compound (III), it is advantageous, in order to remove possible byproducts, to recycle only part of this fraction and to replace the other part with fresh pyridine compound (III).

In a preferred embodiment of the batchwise preparation of the chlorocarbonyl chlorides (I), the total amount of the corresponding lactone (II), the pyridine compound (III) and, if appropriate, a solvent (for example the corresponding chlorocarbonyl chloride target product) are initially charged in a stirred vessel. The reaction system is then heated to a temperature of at least 100° C. or above and, at atmospheric pressure, the simultaneous introduction of hydrogen chloride and phosgene is started. Alternatively, it is also possible to start with the introduction of hydrogen chloride first, and to add phosgene only subsequently thereto. The coproducts formed, carbon dioxide and hydrogen chloride, are removed. After the desired amount of phosgene and, in parallel, hydrogen chloride have been added, the reaction solution is maintained at the resulting temperature for some more time, to allow the after-reaction to take place. During the after-reaction, more hydrogen chloride may be introduced, if desired. To remove the excess phosgene and its coproducts carbon dioxide and hydrogen chloride from the reaction solution, or to reduce their concentration, it is then possible to pass inert gas through the solution (stripping). The resulting reaction solution is finally worked up. In general, work-up is carried out by distillation, if appropriate under reduced pressure. In the case of high-molecular-weight chlorocarbonyl chlorides, other purification processes, such as, for example, crystallization, are also possible.

In a general embodiment of the continuous preparation of the chlorocarbonyl chlorides (I), a solvent (for example the corresponding chlorocarbonyl chloride target product) and the pyridine compound (III) are initially charged in a reactor, for example a stirred tank, the system is heated to the desired temperature of at least 60° C. and gaseous hydrogen chloride is added. Subsequently, at a temperature of at least 100° C., the continuous introduction of lactone (II), which generally contains further pyridine compound (III) and which may, if appropriate, be dissolved in a solvent, and the continuous addition of liquid or gaseous phosgene is started. In general, more hydrogen chloride is introduced in parallel. After the reactor content has been converted into the chlorocarbonyl chloride, the amounts of lactone (II) and phosgene are adjusted such that both are introduced in essentially equimolar amounts. An amount of the reaction volume which corresponds to the amount that is added is discharged from the reaction apparatus, for example via a means to maintain the level or via an overflow.

The reaction solution that has been removed is collected in a downstream container, for example a stirred tank, for after-reaction. During the after-reaction, more hydrogen chloride may be introduced, if desired. Once the downstream container, too, has been filled by the reaction discharge, the overflow is, if required, freed from the coproducts carbon dioxide and hydrogen chloride, as described above, and worked up. Work-up can be carried out, for example, by distillation.

Using the process according to the invention, it is possible to 0.5 prepare chlorocarbonyl chlorides, in particular 5-chlorovaleryl chlorides and derivatives thereof, by phosgenation of the corresponding lactones, in high yield and high purity of more than 98%. The formation of oligomeric and polymeric byproducts is reduced considerably which, in addition to the high yield mentioned, has the further advantage that the amount of residue which has to be disposed of is reduced considerably. Since the viscosity of the reaction mixture no longer, or to a greatly reduced extent, has a tendency to increase, the safety risk owing to blocking of apparatus and pipelines during synthesis and work-up is likewise reduced considerably. A safe operation of the reaction is thus ensured. Furthermore, the tendency to form foam during the synthesis is reduced considerably.

### EXAMPLES

### Example 1 (According to the Invention)

350 kg of δ-valerolactone (3.5 kmol) and 16 kg of β-picoline (3-methylpyridine, 0.2 kmol) were initially charged in a 630 l steel/enamel tank and heated to 120° C. At 120° C., introduction of gaseous hydrogen chloride at 1 m³/h was started, and the temperature was increased further. When 140° C. had been reached, the additional introduction of gaseous phosgene was started, and a total of 380 kg of phosgene gas (3.84 kmol) were introduced over a period of 24 hours. During the introduction, the temperature increased to 145° C. After the addition of phosgene had ended, hydrogen chloride was fed in for a further 3 hours for the after-reaction. The mixture was then cooled to 80° C. and excess phosgene was stripped for 12 hours, using 2.5 m³/h of nitrogen. From the crude discharge, initially a prerun of about 20 l was drawn off at 1 kPa abs (10 mbar abs) and a top temperature of 85° C., and the remainder was then subjected to fractional distillation. This gave 440 kg of 5-chlorovaleryl chloride (2.84 kmol) of a purity of >98 GC area %, which corresponds to a yield of 81%.

### Experimental arrangement for Examples 2 to 8

The experimental arrangement included a 1 l double-jacketed glass vessel with a stirrer, a thermostat, one inlet tube each for the gaseous hydrogen chloride and the phosgene and a two-stage battery of condensers. The two-stage battery of condensers consisted of a high-efficiency condenser, which was kept at a temperature of −10° C., and a dry-ice condenser, which was kept at a temperature of −78° C. The experiments were carried out under atmospheric pressure.

### Example 2 (Comparative Example)

200 g of δ-valerolactone (2 mol) and 8.1 g of 3-methylpyridine (β-picoline, 0.1 mol) were slowly heated with stirring. At a temperature of 50° C., introduction of 10 Nl/h of hydrogen chloride was started, and the temperature was increased further. When 145° C. had been reached, the additional introduction of phosgene was started. At 145-150° C., a total of 46 g of gaseous hydrogen chloride (1.26 mol) and 102 g of gaseous phosgene (1.03 mol) were introduced over a period of 2 hours. During the introduction, a considerable increase in viscosity was observed. To prevent the glass stirrer breaking off, the experiment had to be terminated.

### Example 3 (According to the Invention)

200 g of δ-valerolactone (2 mol) and 8.1 g of β-picoline (3-methylpyridine, 0.1 mol) were slowly heated with stirring. At a temperature of 80° C., introduction of 10 Nl/h of hydrogen chloride was started, and the temperature was increased further. When 140° C. had been reached, the additional introduction of phosgene was started. At 140-150° C., a total of 60 g of gaseous hydrogen chloride (1.64 mol) and 193 g of gaseous phosgene (1.95 mol) were introduced over a period of 4 hours and 45 minutes. When the introduction of gas had ended, the reaction mixture was, for the after-reaction, kept stirring at 145° C. for 1 hour. Excess phosgene was then stripped by passing through nitrogen, and the reaction mixture was subjected to fractional distillation. This gave 205 g of 5-chlorovaleryl chloride (1.32 mol) of a purity of >98 GC area %, which corresponds to a yield of 66%.

### Example 4 (According to the Invention)

200 g of δ-valerolactone (2 mol) and 8.1 g of β-picoline (3-methylpyridine, 0.1 mol) were slowly heated with stirring. At a temperature of 110° C., introduction of 10 Nl/h of hydrogen chloride was started, and the temperature was increased further. When 142° C. had been reached, the additional introduction of phosgene was started. At 142-150° C., a total of 70 g of gaseous hydrogen chloride (1.92 mol) and 202 g of gaseous phosgene (2.04 mol) were introduced over a period of 4 hours and 15 minutes. When the introduction of gas had ended, the reaction mixture was, for the after-reaction, kept stirring at 145° C. for 1 hour. Excess phosgene was then stripped by passing through nitrogen, and the reaction mixture was subjected to fractional distillation. This gave 250 g of 5-chlorovaleryl chloride (1.61 mol) of a purity of >98 GC area %, which corresponds to a yield of 81%.

### Example 5 (According to the Invention)

200 g of δ-valerolactone (2 mol) and 8.1 g of δ-picoline (3-methylpyridine, 0.1 mol) were slowly heated with stirring. At a temperature of 138° C., simultaneous introduction of hydrogen chloride and phosgene was started. At 138-150° C., a total of 61 g of gaseous hydrogen chloride (1.67 mol) and 207 g of gaseous phosgene (2.09 mol) were introduced over a period of 3 hours and 15 minutes. When the introduction of gas had ended, the reaction mixture was, for the after-reaction, kept stirring at 145° C. for 1 hour. Excess phosgene was then stripped by passing through nitrogen, and the reaction mixture was subjected to fractional distillation. This gave 270 g of 5-chlorovaleryl chloride (1.74 mol) of a purity of >98 GC area %, which corresponds to a yield of 87%.

### Example 6 (Comparative Example)

200 g of δ-valerolactone (2 mol) and 34.8 g of Cyanex® 923 (commercial product from Cytec Industries, mixture of various trialkylphosphine oxides with an average molecular weight of 348 g/mol, 0.1 mol) were slowly heated with stirring. At a temperature of 120° C., introduction of 10 Nl/h of hydrogen chloride was started, and the temperature was increased further. When 146° C. had been reached, the additional introduction of phosgene was started. At 146-150° C., a total of 44 g of gaseous hydrogen chloride (1.21 mol) and 90 g of gaseous phosgene (0.91 mol) were introduced over a period of 3 hours. During the introduction, a considerable increase in viscosity was observed. To prevent the glass stirrer from breaking off, the experiment had to be terminated.

### Example 7 (Comparative Example)

200 g of δ-valerolactone (2 mol) and 12.8 g of dimethylpropyleneurea (1,3-dimethyltetrahydro-2(1H)-pyrimidinone, 0.1 mol) were slowly heated with stirring. At a temperature of 100° C., introduction of 10 Nl/h of hydrogen chloride was started, and the temperature was increased further. When 145° C. had been reached, the additional introduction of phosgene was started. At 145-150° C., a total of 6 g of gaseous hydrogen chloride (0.16 mol) and 7 g of gaseous phosgene (0.07 mol) were introduced over a period of 25 minutes. During the introduction, a considerable increase in viscosity was observed. To prevent the glass stirrer from breaking off, the experiment had to be terminated.

### Example 8 (Comparative Example)

200 g of δ-valerolactone (2 mol) and 12.8 g of dimethylpropyleneurea (1,3-dimethyltetrahydro-2(1H)-pyrimidinone, 0.1 mol) were slowly heated with stirring. At a temperature of 138° C., simultaneous introduction of hydrogen chloride and phosgene was started. At 145-150° C., a total of 67 g of gaseous hydrogen chloride (1.84 mol) and 210 g of gaseous phosgene (2.12 mol) were introduced over a period of 3 hours. When the introduction of gas had ended, the reaction mixture was, for the after-reaction, kept stirring at 145° C. for 1 hour. Excess phosgene was then stripped by passing through nitrogen, and the reaction mixture was subjected to fractional distillation. This gave 248 g of 5-chlorovaleryl chloride (1.60 mol) of a purity of >98 GC area %, which corresponds to a yield of 80%.

### Example 9 (Comparative Example)

192 g of γ-butyrolactone (2.23 mol) and 2 g of pyridine (0.025 mol) were initially charged in a double-jacketed glass vessel and heated to 120° C. With vigorous stirring, a total of 60 g of gaseous phosgene (0.61 mol) were introduced at 120-124° C. over a period of 8 hours. The remaining unreacted phosgene was stripped with nitrogen and the crude discharge was then subjected to fractional distillation. The first fraction of 76 g contained 21.6 GC area % of 4-chlorobutyryl chloride, and the second fraction of 110 g contained 2.6 GC area % of 4-chlorobutyryl chloride, which corresponds to a total yield of 6%. The experimental data are summarized in Table 1. Example 2 shows that, when introduction of hydrogen chloride was started below the temperature limit according to the invention, the reaction had to be terminated owing to a considerable increase in viscosity. Examples 3 to 5 according to the invention show that the yield of 5-chlorovaleryl chloride increases considerably when the start temperature for the introduction of hydrogen chloride is increased. When the introduction of hydrogen chloride was started at 138° C., a yield of 87% could be realized.

Whereas, in Example 4 according to the invention, with β-picoline as catalyst and the introduction of hydrogen chloride being started at 110° C., it was possible to obtain a yield of 5-chlorovaleryl chloride of 81%, the use of trialkylphosphine oxides and dimethylpropyleneurea as catalyst (see Comparative Examples 6 and 7) results in a considerable increase in the viscosity of the reaction solution. In both cases, the reaction had to be terminated.

Example 3 according to the invention shows that when β-picoline is used as starting material a liquid reactor discharge is obtained even at an inlet temperature of the hydrogen chloride gas of considerably below 100° C., whereas, on using trialkylphosphine oxides and dimethylpropyleneurea as catalyst (see Comparative Examples 6 and 7), the reaction had to be terminated even at 120° C. and 100° C., respectively, owing to a viscous reactor content.

As shown by the comparison between Example 5 according to the invention and Comparative Example 8, it is possible even when catalysts not according to the invention are used, such as dimethylpropyleneurea, to achieve a certain improvement by increasing the start temperature for the introduction of hydrogen chloride; however, this is far from the excellent result of the process according to the invention. Under otherwise comparable conditions, Example 5 according to the invention gave an about 9 rel.-% higher yield.

Even if a liquid reactor discharge is obtained by the use of catalysts not according to the invention, such as dimethylpropyleneurea, at a higher start temperature of 138° C. for the introduction of hydrogen chloride, as shown by Comparative Example 8, there is a risk of foam-forming and of an increase in viscosity up to the reactor content solidifying if the reactor heating fails. The use of the pyridine compounds (III) according to the invention is superior to the use of catalysts not according to the invention, such as, for example, urea compounds of phosphine oxides.

Comparative example 9 shows that even if pyridine compounds (iii) are used, without the introduction of hydrogen chloride an only insufficient yield is obtained.

TABLE 1

| | | Start of | | | | |
|---|---|---|---|---|---|---|
| | | HCl | COCl₂- | Course of the | | |
| Example | Catalyst | introduction | Introduction | reaction | Yield | Purity |
| 1 | β-picoline | 120° C. | 140 to 145° C. | liquid discharge | 81% | >98% |
| 2* | β-picoline | 50° C. | 145 to 150° C. | terminated | — | — |
| | | | | owing to high | | |
| | | | | viscosity | | |
| 3 | β-picoline | 80° C. | 140 to 150° C. | liquid discharge | 66% | >98% |
| 4 | β-picoline | 110° C. | 142 to 150° C. | liquid discharge | 81% | >98% |
| 5 | β-picoline | 138° C. | 138 to 150° C. | liquid discharge | 87% | >98% |
| 6* | Cyanex ® 923 | 120° C. | 146 to 150° C. | terminated | — | — |
| | (trialkylphosphine | | | owing to high | | |
| | oxides ) | | | viscosity | | |
| 7* | dimethylpropylene- | 100° C. | 145 to 150° C. | terminated | — | — |
| | urea | | | owing to high | | |
| | | | | viscosity | | |
| 8* | dimethylpropylene- | 138° C. | 145 to 150° C. | liquid discharge | 80% | >98% |
| | urea | | | | | |
| 9* | pyridine | without | 120 to 124° C. | liquid discharge | 6% | not |
| | | | | | | determined |

*Comparative Example

We claim:

1. A process for preparing chlorocarbonyl chlorides of the formula (I) 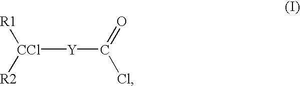
in which
R¹ and R² independently of one another
are a hydrogen atom, a carbon-containing organic radical, a halogen, a nitro or a cyano group,
and Y
is an alkylene chain having 1 to 10 carbon atoms in the chain, which is unsubstituted or substituted by carbon-containing organic radicals, halogen, nitro and/or cyano groups, where the alkylene chain may be interrupted by an ether, a thioether, a tertiary amino or a keto group,
where the carbon-containing organic radicals of Y and/or R¹ and/or R² may be attached to one another forming a nonaromatic system, by reacting a lactone of the formula (II) 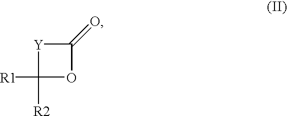
in which R¹, R² and Y are as defined above, with hydrogen chloride and phosgene in the presence of a catalyst, which comprises introducing hydrogen chloride before and/or during the addition of phosgene, where the introduction of hydrogen chloride is started only when a temperature of at least 60° C. has been reached and a pyridine compound is used as catalyst.

2. A process as claimed in claim 1, wherein the catalyst is employed in a concentration of 0.1-20 mol %, based on the lactone (II).

3. A process as claimed in claim 1 or 2, wherein the catalyst used is 3-methylpyridine (β-picoline).

4. A process as claimed in any of claims 1 to 3, wherein a total of 0.5-2 mol of hydrogen chloride are employed per mole of lactone (II).

5. A process as claimed in any of claims 1 to 4, wherein the introduction of hydrogen chloride is started only when a temperature of at least 100° C. has been reached.

6. A process as claimed in any of claims 1 to 5, wherein the reaction with phosgene is carried out at a temperature of 100-200° C. and an absolute pressure of 0.01-5 MPa.

7. A process as claimed in any of claims 1 to 6, wherein 5-chlorovaleryl chloride or derivatives thereof are prepared.

8. A process as claimed in any of claims 1 to 7, wherein 5-chlorovaleryl chloride is prepared.
